(19) United States
(12) Patent Application Publication
(10) Pub. No.: US 2016/0000749 A1
(43) Pub. Date: Jan. 7, 2016

(54) Method of Inhibiting or Treating a Dengue Virus Infection with Quercetin

(71) Applicant: Universiti Malaya, W.P. Kuala Lumpur (MY)

(72) Inventor: Sazaly Abu Bakar, Kuala Lumpur (MY)

(21) Appl. No.: 14/854,194

(22) Filed: Sep. 15, 2015

Related U.S. Application Data
(63) Continuation of application No. 14/384,373, filed on Sep. 10, 2014.

(30) Foreign Application Priority Data
Mar. 30, 2012 (MY) PI2012700140

(51) Int. Cl. A61K 31/352

(52) U.S. Cl. CPC A61K 31/352

(57) ABSTRACT

The present invention relates to a composition having antiviral activity for prophylaxis or treatment of flavivirus infection or a disease resulting therefrom in humans or animals, characterised in that the composition comprises quercetin, or analogues, or derivatives thereof. The composition may further comprise a pharmaceutically acceptable carrier. The antiviral activity may include inhibition of virus attachment to host cells and inhibition of intracellular virus replication. The flavivirus may comprise dengue virus type-1, dengue virus type-2, dengue virus type-3, and dengue virus type-4.

### BACKGROUND OF THE INVENTION

1. Field of the Invention

This invention relates to a composition having antiviral activity, and more particularly to a composition comprising flavonoid quercetin having antiviral activity against flavivirus disease.

2. Description of Related Arts

Dengue virus (DENV) is a member of the genus Flavivirus of the family Flaviviridae. The virus is a significant human pathogen which causes a wide spectrum of clinical illnesses ranging from a silent or mild febrile infection, self-limited dengue fever (DF) to the severe dengue hemorrhagic fever (DHF) and dengue shock syndrome (DSS). There are four genotypes of dengue virus; DENV-1, DENV-2, DENV-3, and DENV-4. All four viruses cause similar disease with no different clinical presentation. The virus is transmitted to humans by two species of mosquitoes, Aedes aegypti and Aedes albopictus.

Dengue is endemic in most tropical and subtropical areas of the world and causing many deaths. It is rapidly spreading to many other parts of the world threatening at least 2.5 million people annually. Currently there is no effective vaccine or antiviral available for the disease. Since vaccine for dengue is difficult and complex to develop it is critical that an effective antiviral is discovered or developed.

Plant and plant-derived compounds remain an important source for the discovery and development of new antiviral drugs. This is because plants in general offer many natural compounds that may potentially be beneficial as antivirals with possible low side effects.

U.S. Pat. No. 6,638,514 B1 disclosed vaccine compositions of attenuated dengue virus. The cited art provides methods for stimulating the immune system of an individual to induce protection against all four dengue virus serotypes by administration of attenuated dengue-1, dengue-2, dengue-3, and dengue-4 virus. As the composition introduced in the cited art is a vaccine composition, there is a possibility of developing side effects and vaccine being less ineffective against the virus. Therefore, there is a need to find an alternative by using natural compound that has antiviral activity and works for prophylaxis or treatment of Flavivirus infection or a disease.

U.S. Pat. No. 7,473,424 B2 disclosed an anti-Dengue virus antibody, including isolated nucleic acids that encode at least one anti-Dengue virus antibody, vectors, host cells, transgenic animals or plants, and methods of making and using thereof, including therapeutic compositions, methods, and devices. However, a need has arisen to search for natural compound as an alternative to the use of an antibody for the treatment of Flavivirus diseases including, but not limited to, dengue virus.

US Patent Application No. 2010/0331337 A1 disclosed a composition containing quercetin, vitamin B3, vitamin C, and folic acid for enhancing physical or mental performance or treating various diseases or disorders. The various diseases do not include Flavivirus infection or diseases. Hence, there is a potential to have a composition comprising quercetin for treatment of Flavivirus infection or a disease resulting therefrom.

US Patent Application No. 2008/0026076 A1 disclosed a nutritional supplement comprising quercetin for improving cardiovascular health. There is no disclosure on the use of quercetin on treating flavivirus disease. Therefore, there is a need to improve cited art on having a composition having quercetin and possibly with a pharmaceutically acceptable carrier for the purpose of treating flavivirus diseases including dengue virus.

Accordingly, it can be seen in the prior arts that there exists a need to provide a composition for prophylaxis or treatment of flavivirus infection or a disease resulting therefrom in humans or animals.

### SUMMARY OF INVENTION

It is an objective of the present invention to provide a composition having antiviral activity against Flavivirus.

It is also an objective of the present invention to provide a composition comprising quercetin, a plant-derived compound, having antiviral activity against Flavivirus.

It is yet another objective of the present invention to provide a composition comprising quercetin as the main active component and pharmaceutically acceptable carrier having antiviral activity against dengue virus.

Accordingly, these objectives may be achieved by following the teachings of the present invention. The present invention relates to a composition having antiviral activity for prophylaxis or treatment of flavivirus infection or a disease resulting therefrom in humans or animals, characterised in that the composition comprises quercetin, or analogues, or derivatives thereof. The composition may further comprise a pharmaceutically acceptable carrier. The flavivirus may comprise dengue virus including dengue virus type-1, dengue virus type-2, dengue virus type-3, and dengue virus type-4.

### BRIEF DESCRIPTION OF THE DRAWINGS

The features of the invention will be more readily understood and appreciated from the following detailed description when read in conjunction with the accompanying drawings of the preferred embodiment of the present invention, in which:

FIG. 1 is a chemical formula structure of a quercetin.

FIG. 2 is a graph showing cytotoxicity effects of quercetin on Vero cells.

FIG. 3(a) is a graph showing anti-viral properties of quercetin against DENV-2 replication calculated using foci forming unit reduction assay (FFURA). Quercetin was added to cells after virus adsorption.

FIG. 3(b) is a graph showing anti-viral properties of quercetin against DENV-2 replication determined using quantitative real time polymerase chain reaction amplification (qRT-PCR). Quercetin was added to cells after virus adsorption.

FIG. 4(a) is a graph showing anti-viral effects of continuous treatment with quercetin against DENV-2 replication calculated using foci forming unit reduction assay (FFURA). Quercetin was added to cells 5 hours before infection and continuously present during the 4 days infection.

FIG. 4(b) is a graph showing anti-viral effects of continuous treatment with quercetin against DENV-2 replication determined using quantitative real time polymerase chain reaction amplification (qRT-PCR). Quercetin was added to cells 5 hours before infection and continuously present during the 4 days infection.

FIG. 5(a) is a graph showing anti-adsorption activity of quercetin, against DENV-2 calculated using foci forming unit reduction assay (FFURA). Quercetin was added to cells simultaneously during infection.

FIG. 5(b) is a graph showing anti-adsorption activity of quercetin against DENV-2 and the respective DENV-2 RNA copy number quantified using qRT-PCR. Quercetin was added to cells simultaneously during infection.

FIG. 6(a) is a picture showing foci size of dengue infected cells not treated with quercetin.

FIG. 6(b) is a picture showing foci size reduction in DENV-infected cells treated with quercetin.

### DETAILED DESCRIPTION OF THE INVENTION

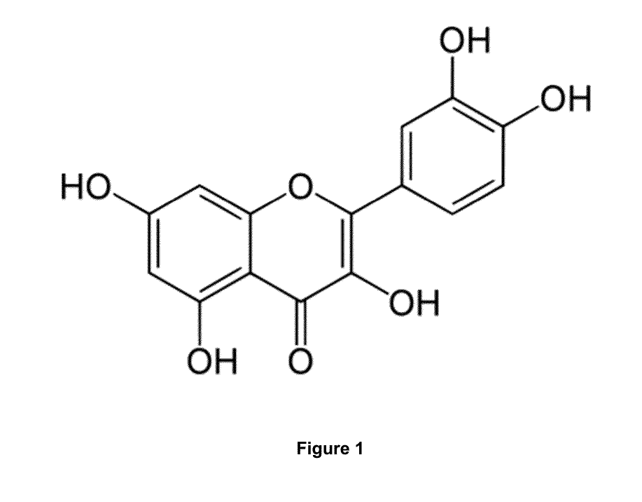

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting but merely as a basis for claims. It should be understood that the drawings and detailed description thereto are not intended to limit the invention to the particular form disclosed, but on the contrary, the invention is to cover all modification, equivalents and alternatives falling within the scope of the present invention as defined by the appended claims. As used throughout this application, the word “may” is used in a permissive sense (i.e., meaning having the potential to), rather than the mandatory sense (i.e., meaning must). Similarly, the words “include,” “including,” and “includes” mean including, but not limited to. Further, the words “a” or “an” mean “at least one” and the word “plurality” means one or more, unless otherwise mentioned. Where the abbreviations of technical terms are used, these indicate the commonly accepted meanings as known in the technical field. For ease of reference, common reference numerals will be used throughout the figures when referring to the same or similar features common to the figures. The present invention will now be described with reference to FIG. 1.

The present invention relates to a composition having antiviral activity for prophylaxis or treatment of flavivirus infection or a disease resulting therefrom in humans or animals, characterised in that the composition comprises quercetin, or analogues, or derivatives thereof. The terms “analogues or derivatives” as used herein also includes salts, solvates, hydrates, prodrugs, and isomers including tautomers and stereoisomers of quercetin.

In a preferred embodiment of the present invention, said quercetin is in a concentration range of about 0.1 to 100% by weight.

In a preferred embodiment of the present invention, the effective concentration of said composition is in a range of 29 μg/ml (28.9 μg/ml)-35 μg/ml (35.7 μg/ml) of quercetin.

In a preferred embodiment of the present invention, said composition formulated with a pharmaceutically acceptable carrier.

In a preferred embodiment of the present invention, the effective concentration of said composition having antiviral activity is 35.7 μg/ml of quercetin.

In a preferred embodiment of the present invention, the antiviral activity is inhibition and reduction of virus replication.

In a preferred embodiment of the present invention, flavivirus is selected from a group comprising dengue virus type-1, dengue virus type-2, dengue virus type-3, and dengue virus type-4.

In a preferred embodiment of the present invention, said composition is for the prophylaxis or treatment of dengue virus type-2.

In the present invention, quercetin may be obtained by chemical synthesis or extracted from a plant.

A person skilled in the art may attempt to modify the molecular structure of quercetin for the purpose of the present invention. It is possible for such modification by adding, removing, or substituting moieties in the quercetin compound. Said quercetin may be modified by means of esterification, hydrolysis, saponification, substitution in one or more positions and any other chemical reaction modifications.

FIG. 1 shows the chemical formula structure of quercetin [2-(3,4-dihydroxyphenyl)-3,5,7-trihydroxy-4H-1-benzopyran-4-one].

### Compound Quercetin

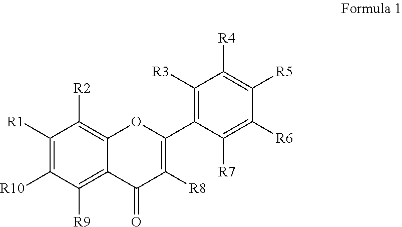

In a preferred embodiment, quercetin according to formula 1, or an analogue or derivative thereof, according to formula 1, including salts, solvates, hydrates, prodrugs, and isomers including tautomers or stereoisomers thereof, wherein:

R₁, R₂, R₃, R₄, R₅, R₆, R₇, R₈, R₉, and R₁₀, which are each independently selected from the group consisting of: —H, —OH, —OR′, —SH, —SR′, —SOR′, —NO₂, —NH₂, —NHR′, —N(R′)₂, —NHCOR′,—N(COR′)₂, —NHSO₂R′, —CN, halogen, —C(═O)H, —C(═O)R′, —CO₂H, —CO₂R′, alkyl, substituted alkyl, alkenyl, substituted alkenyl, cycloalkenyl, substituted cycloalkenyl, alkynyl, substituted alkynyl, aryl, substituted aryl, heterocyclyl, substituted heterocyclyl, heteroaryl and substituted heteroaryl;

wherein each substituted alkyl, substituted cycloalkyl, substituted alkenyl, substituted cycloalkenyl, substituted alkynyl, substituted aryl, substituted heteroaryl, and/or substituted heterocyclyl has 1-3 substituents each independently selected from the group consisting of:

—OH, —OR′, —SH, —SR′, —SOR′, —SO₂R′, —NO₂, —NH₂, —NHR′, —N(R′)₂, —NHCOR′, —N(COR′)₂, —NHSO₂R′, —CN, halogen, —C(═O)H, —C(═O)R′, —CO₂H, —CO₂R′, alkyl, alkyl substituted with 1-3 R″, alkenyl, alkenyl substituted with 1-3 R″, cycloalkenyl, cycloalkenyl substituted with 1-3 R″, alkynyl, alkynyl substituted with 1-3 R″, aryl, aryl substituted with 1-3 R″, heterocyclyl, heterocyclyl substituted with 1-3 R″, heteroaryl and heteroaryl substituted with 1-3 R″;

wherein each R′ is independently selected from the group consisting of alkyl, alkyl substituted with 1-3 R″, cycloalkyl, cycloalkyl substituted with 1-3 R″, alkenyl, alkenyl substituted with 1-3 R″, cycloalkenyl, cycloalkenyl substituted with 1-3 R″, alkynyl, alkynyl substituted with 1-3 R″, aryl, aryl substituted with 1-3 R″, alkylaryl, alkylaryl substituted with 1-3 R″, heterocyclyl, heterocyclyl substituted with 1-3 R″, heteroaryl and heteroaryl substituted with 1-3 R″; and

wherein each R″ is independently selected from the group consisting of: —OH, —SH, —NO₂, —NH₂, —CN, halogen, —C(═O)H, and —CO₂H.

In a preferred embodiment, the alkyl is methyl, ethyl, n-propyl, iso-propyl, n-butyl, iso-butyl, sec-butyl, tert-butyl, n-pentyl, 1-methylbutyl, 2-methylbutyl, 3-methylbutyl or 2,2′-dimethylpropyl. In a preferred embodiment, the aryl comprises 4-10 carbon atoms. In a preferred embodiment, the cycloalkyl comprises 3-6 carbon atoms.

In a preferred embodiment, the R₁, R₄, R₅, R₈ and R₉ are —OH.

In a preferred embodiment, the R₂, R₃, R₆, R₇, and R₁₀ are —H.

Quercetin has a molecular formula of C₁₅H₁₀O₇, as shown in FIG. 1.

While it may be possible for the quercetin to be administered alone, it is preferable to present it with the pharmaceutically acceptable carrier. Said carrier(s) optimally are acceptable in the sense of being compatible with other ingredients or compounds of said composition and not deleterious for any administration routes including oral, rectal, nasal, topical, vaginal, or parenteral administration.

In a preferred embodiment, the pharmaceutically acceptable carrier comprises water, solvents, pH buffering agents, stabilisers, excipients, diluents, or mixtures thereof. As used herein, the term “pharmaceutically acceptable carrier” means inert, non-toxic solid or liquid filler, diluent or encapsulating material, not reacting with the active ingredients, which according to the present invention. These carriers are known to the man versed in the art. Wetting agents and emulsifiers, as well as release agents, coating agents, and preservatives may also be present in the preparations of the present invention. The amount of quercetin that may be combined with the carrier materials to produce a single dosage form will vary, depending upon the patient treated and the particular mode of administration.

The term “pharmaceutically acceptable carrier” herein also includes food additives generally used in foods and drinks, such as a sweetener, a colouring agent, a preservative, a thickening stabiliser, an antioxidant, a colour developing agent, a bleaching agent, a bitter agent, an enzyme, a sour agent, a seasoning, a nutrient supplement, an enzyme, a manufacture facilitating agent, and a flavour. Said composition may also be manufactured in a product, particularly food product for example juice drink, milk, coffee, chocolates, and snack bars.

The composition of the present invention formulated with a pharmaceutically acceptable carrier may be prepared in any appropriate manner, for instance by homogenously mixing, coating and/or grinding the active ingredients, in a one-step or multi-step procedure.

Said composition may conveniently be presented in unit dosage form and may be prepared by any method in the art of pharmacy. Such methods may include for example, homogenising said composition with a chosen carrier before shaping product of the homogenisation into a unit dosage form such as cream.

In a preferred embodiment of the present invention, said composition can be formulated for any suitable route of administration, depending on whether local or systemic treatment is desired and which area is to be treated. Said composition may be prepared and formulated for parenteral administration, such as intravenous, intraperitoneal, intramuscular, or subcutaneous injection. Said composition of the present invention may also be prepared and formulated in a conventional form either as liquid solution or emulsion, or solid form for solubilising in liquid, which is suitable for injection. Said parental administration may involve preparation including the use of sterile aqueous or non-aqueous solutions, and emulsions. Some examples of non-aqueous solvents that could be used in formulating said composition of the present invention are propylene polyethylene glycol, vegetable oils, and injectable organic esters. Aqueous carriers that could be used in formulating said composition of the present invention may include water.

Said composition of the present invention may also be suitable for oral administration, which may be presented as capsules or tablets, each containing a predetermined amount of quercetin; as a powder or granules; as solution; or as an oil-in-water liquid emulsion.

A person skilled in the art can easily determine appropriate dose, schedule, and method of administration for the exact formulation of the composition being used, in order to achieve what is desired as the “effective amount” in an individual patient. Said dose may vary depending on the mode of administration, age, and body weight of a patient, the symptom developed by the patient and the like. The term “effective amount” herein can be defined as, for example as the blood or tissue level desired in the patient that corresponds to a concentration of quercetin of said composition of the present invention. The person skilled in the art can also be readily determine and use an appropriate indicator of the “effective amount” of said composition of the present invention by pharmacological end-point analysis.

Below is an example of quercetin and its activity on dengue virus type-2 (NGC strain) replication in cell culture, from which the advantages of the present invention may be more readily understood. It is to be understood that the following example is for illustrative purpose only and should not be construed to limit the present invention in any way.

### Example

The present example is carried out to examine anti-dengue virus properties of flavonoid quercetin. In this example, quercetin activities on DENV-2 (NGC strain) replication were evaluated in cell culture system. The effects of the compound were evaluated against different stages of dengue virus replication including virus adsorption, intracellular replication and direct virucidal activities.

The bioflavonoid, quercetin obtained from Sigma-Aldrich, St. Louis, Mo., USA was evaluated for its potential activities against dengue virus replication. Dimethyl sulfoxide (DMSO) from Sigma-Aldrich, St. Louis, Mo., USA was used to dissolve the lyophilized form of quercetin and the prepared stock solution (20 mg/mL) was stored at −20° C. Stock solution was diluted using cell culture medium and sterilized by a syringe filter with 0.2 micron pore size (Millipore, Mass., USA) right before each experiment.

C6/36 mosquito cell line derived from Aedes albopictus and Vero cell line were used. Both cell lines were maintained and propagated in Eagle's Minimum Essential Medium (EMEM) containing 10% fetal bovine serum. Cultured C6/36 and Vero cells were incubated at 28° C. and 37° C., respectively in 5% CO₂ humidified chamber. At the time of virus propagation, serum concentration was reduced to 2%. Dengue virus type-2 (DENV-2) New Guinea C strain (NGC) was propagated using C6/36 cell line and harvested after CPE presentation on day seven post infection. After titration, viral stock was stored at −70° C. MTT (Dimethyl thiazolyl diphenyl tetrazolium salt) assay was performed to determine cytotoxicity of quercetin on Vero cells.

Post-adsorption antiviral activity of quercetin against intracellular replication of DENV-2 was performed by inoculating 200 FFU of virus to each well containing monolayer of Vero cells. After adsorption of virus to the cells for one hour at 37° C., the cells were washed with PBS to remove the unadsorped viruses. Then, different concentrations of quercetin were added to the cells followed by four days incubation at 37° C. Viral foci formed were counted and DENV RNA copy number to indicate inhibition of virus replication was quantified using quantitative RT-PCR.

In separate experiment, to determine the antiviral activities of quercetin when used continuously, different concentrations of quercetin were added to the Vero cell monolayer five hours before virus infection. After five hours of pre-infection treatment, the cells were washed twice with sterile PBS and then 200 FFU of DENV-2 was inoculated to the cells and incubated at 37° C. for one hour. Infected cells were continuously incubated with the respective concentration of quercetin for four days post infection. Viral foci formed were counted and DENV RNA copy number to indicate inhibition of virus replication was quantified using quantitative RT-PCR.

In another experiment, Vero cells at ˜80% confluency were infected with 200 FFU of DENV-2 in the presence or absence of different concentrations of quercetin. The microplate was kept at 37° C. for one hour for virus adsorption. Then the cells were washed twice using sterile PBS and incubated for two hours at 37° C. Cells were then incubated for 4 days and foci formed was counted and DENV RNA copy number to indicate inhibition of virus adsorption was quantified using quantitative RT-PCR.

Direct virucidal effect of quercetin was investigated by incubating DENV-2 suspension containing 200 FFU with an equal volume of the different concentrations of quercetin for two hours at 37° C. Then, Vero cells were infected with the treated viral suspension in triplicates. At one hour adsorption at 37° C., cells were washed twice with PBS in order to remove unadsorbed viruses. The microplate was incubated at 37° C. for four days. Viral foci formed were counted and DENV RNA copy number to indicate direct virucidal effects was quantified using quantitative RT-PCR.

In general, antiviral activity of quercein was evaluated by measuring the reduction in number of viral foci. Confluent Vero cell monolayers prepared in 24 wells cell culture microplates were used. Then the infected cells treated using the different treatment regime was overlaid with 1.5% of carboxy methyl cellulose (CMC) (Sigma-Aldrich, St. Louis, Mo., USA) containing EMEM. Viral foci were visualized using peroxidase-based foci staining assay four days post infection. The number of DENV-2 foci formed was counted under a stereomicroscope and the virus titer was expressed as Foci-Forming-Unit (FFU). The percentage of viral foci reduction (RF %) compared to untreated controls was calculated as follows:

RF(%)=(C−T)×100/C
where, C is the mean of the number of foci for negative control well (without compound) and T is the mean of the number of foci in treated wells.

Reduction in the number of viral foci was further verified using quantitative real time polymerase chain reaction (qRT-PCR) amplification.

Quantitative RT-PCR was performed to determine the effect of the compound on DENV replication by quantifying DENV-2 genomic RNA copy number. Briefly, intracellular and extracellular DENV-2 RNAs were harvested from the DENV-infected Vero cells. Viral RNA was extracted using two types of RNA extraction kits (QIAamp Viral RNA mini kit and RNeasy mini kit) (Qiagen, Hilden, Germany). Quantitative RT-PCR assay was performed by adding 1 μl of extracted DENV RNA to the SensiMix SYBR green reagent (Quantace, Watford, United Kingdom) which contained 7.4 μl ddH2O, 10 μl 2× SensiMix One-Step, 0.4 μl 50×SYBR Green solution, 10 units of RNAse Inhibitor, 50 μmol of forward (DNF) and also reverse (D2R) primers. All samples were assayed in triplicate. Amplification was performed using the DNA Engine Opticon system (MJ Research/Bio-Rad, Hercules, Calif.) with the following cycling conditions: reverse transcription at 50° C. for 30 min, initial denaturation at 95° C. for 10 min, followed by 45 cycles of 95° C. for 15 sec, 59° C. for 30 sec and 72° C. for 30 sec. Melting curve analysis was subsequently performed at temperature from 60° C. to 98° C. to verify the assay specificity. For absolute quantitation of the viral RNA, a standard curve was established with a serially diluted RNA extracted from DENV-2 stock with a known titer.

### Results

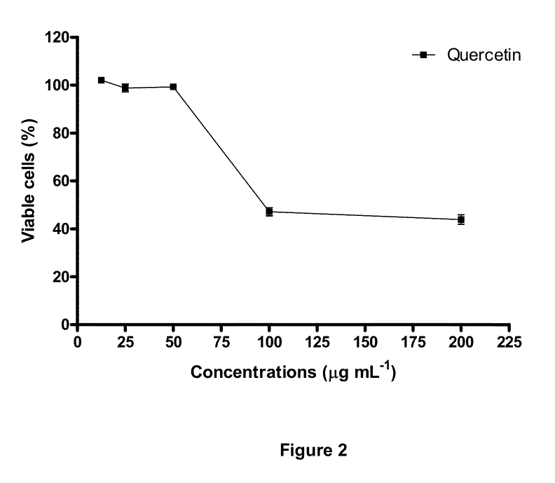

The cytotoxicity effect of quercetin against Vero cells was at CC₅₀ 252.6±0.17 μg/mL (FIG. 2). There was no cytotoxicity effect of the vehicle control, 1% DMSO against Vero cells.

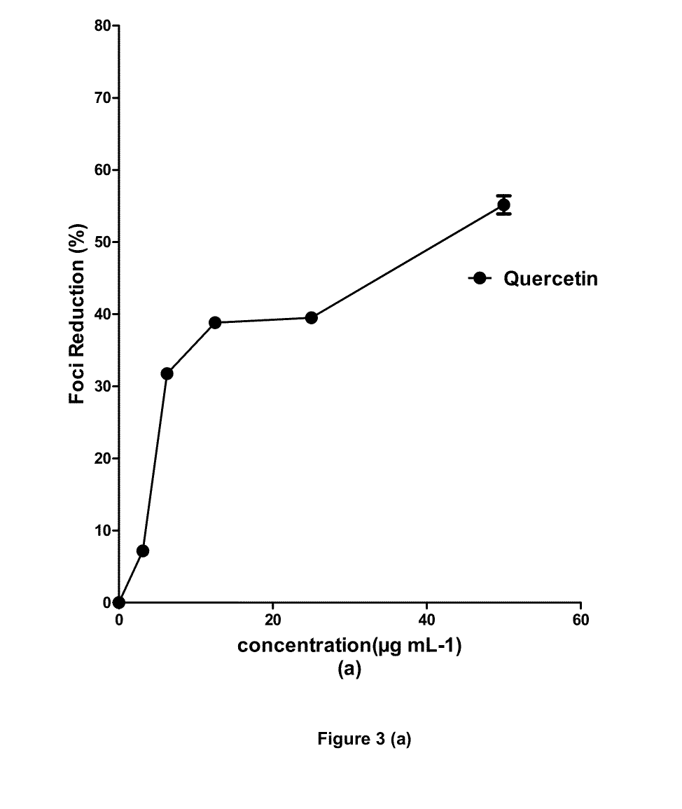

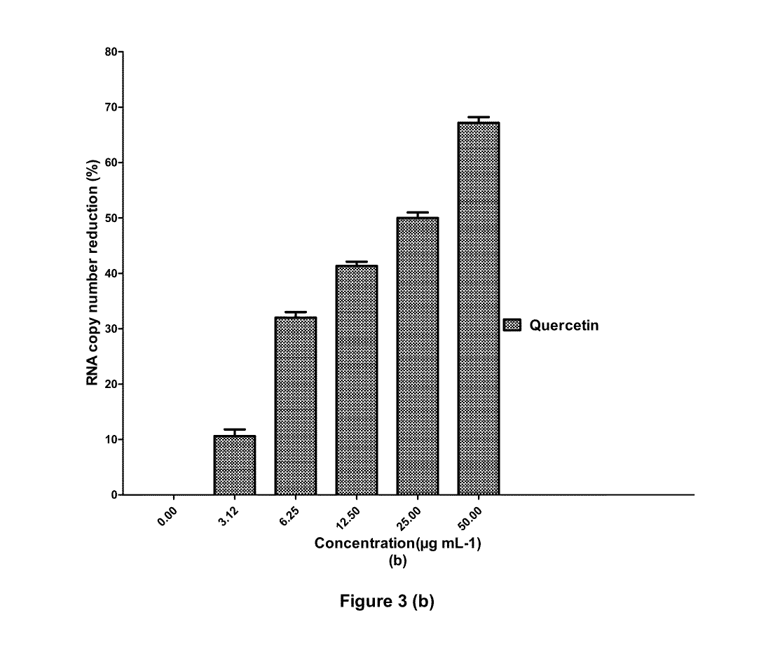

Quercetin exhibited antiviral activity against DENV-2 with IC₅₀=35.7 μg/mL when added post viral adsorption (FIG. 3(a)). The SI value for quercetin in post-adsorption assay was relatively high at 7.07. It was also demonstrated that the level of DENV-2 specific RNA production in the presence of 50 μg/mL quercetin decreased by more than 67%±1 when compared to the non-treated infected cells (FIG. 3(b)).

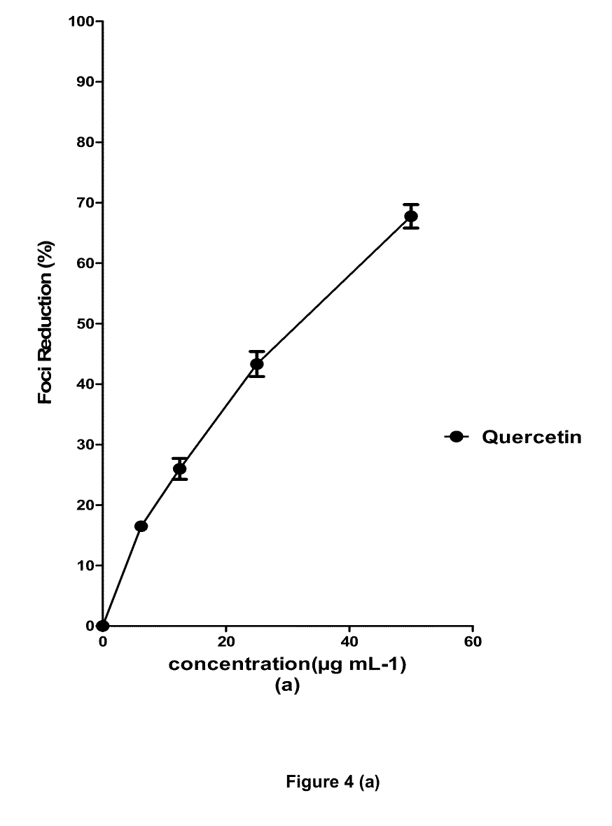

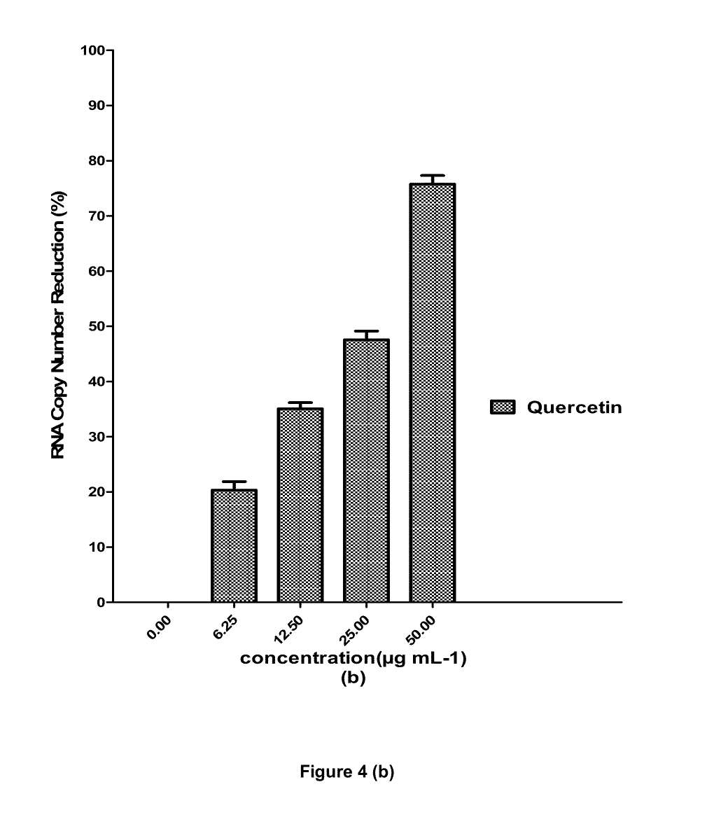

Continuous treatment of cells with quercetin from 5 hours before virus infection and up to 4 days post-infection exhibited anti-dengue activity with IC₅₀=28.9 μg/mL (FIG. 4(a)). The SI value for continuous treatment with quercetin was 8.74 and is higher than the SI value at 7.07 for post-adsorption assay. Continuous treatment of Vero cells with 50 μg/mL quercetin decreased the number of DENV-2 foci by 70%±1.5 when compared to the non-treated cells (FIG. 4 (a). The level DENV-2 RNA production decreased by more than 75.7%±1.57

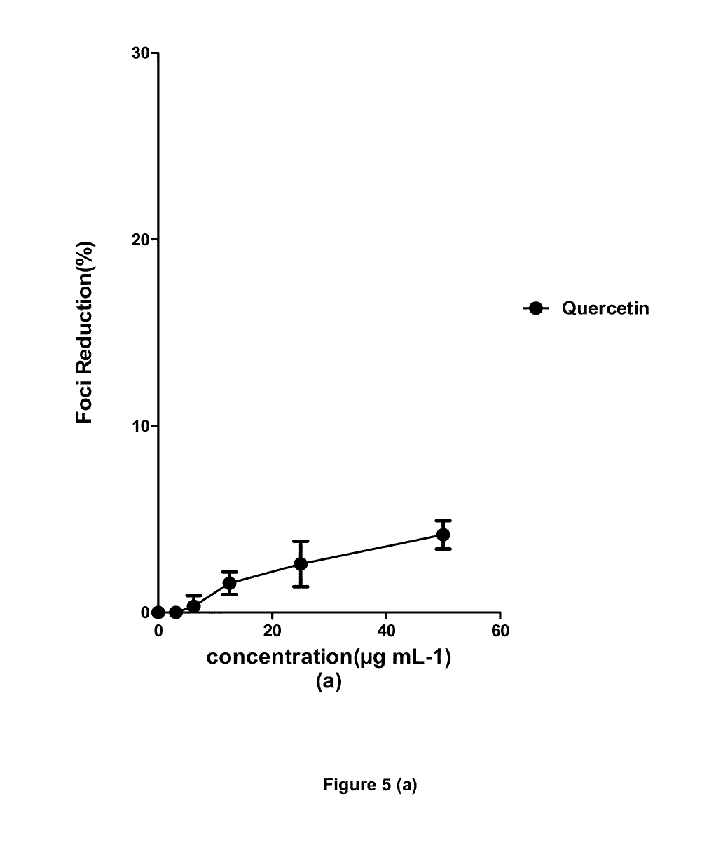

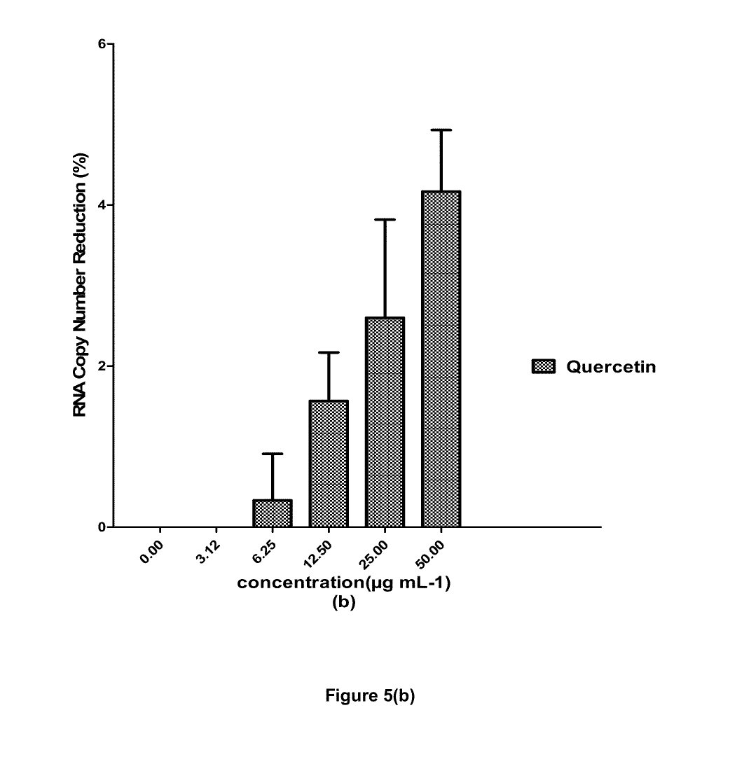

There was ˜5% anti-adsorption activity for quercetin against DENV-2 FIGS. 5(a) and 5(b)).

Quercetin has no significant direct virucidal activity against DENV-2. qRT-PCR analysis showed that there was no significant decrease in copy number of DENV-2 RNA following direct treatment of DENV-2 with the different concentrations of compound.

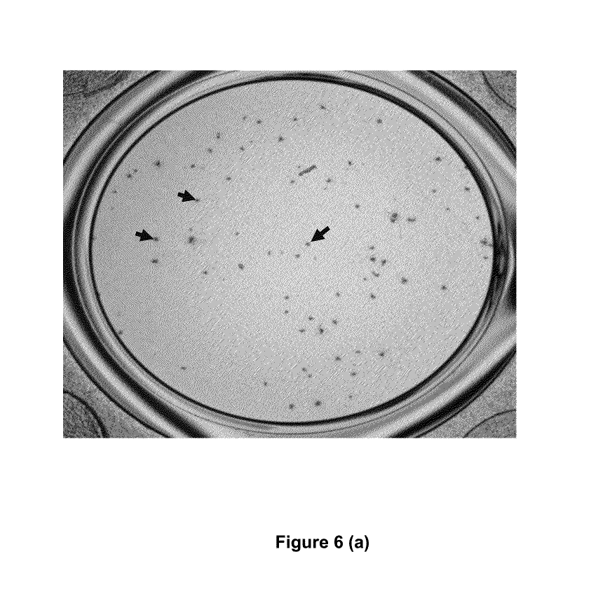

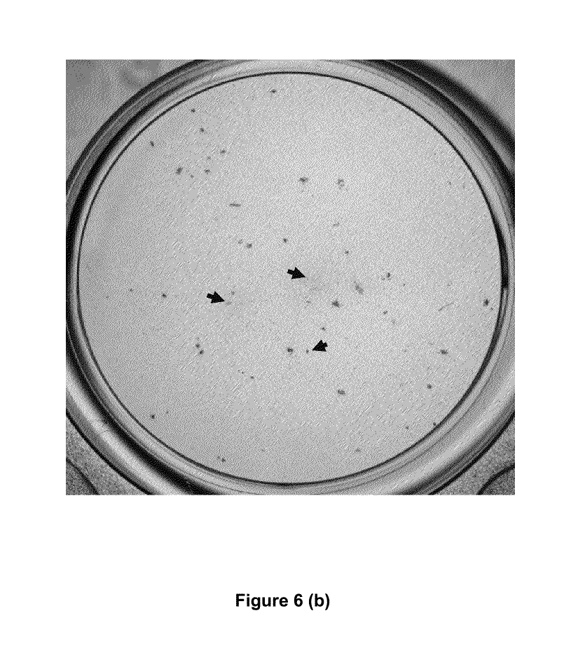

The majority of the viral foci formed in cells treated with 50 μg/ml quercetin appeared small, less intensely stained and more diffused (FIG. 6(b)), compared to the larger, well-defined and more intensely stained foci of the untreated dengue infected cells (referring to FIG. 6(a)). The arrows in FIG. 6(b) indicate foci which are more diffused, less intensely stained and smaller than the foci in non-treated cells. This observation is consistent with the reduction of the percentage of foci and RNA copy number.

It is shown in the present example that quercetin consistently showed significant antiviral activity against DENV-2 in Vero cells when used as antiviral post virus adsorption and when continuously present in cells.

Although the present invention has been described with reference to specific embodiments, also shown in the appended figures, it will be apparent for those skilled in the art that many variations and modifications can be done within the scope of the invention as described in the specification and defined in the following claims.

I/we claim:

1. A composition having antiviral activity for prophylaxis or treatment of flavivirus infection or a disease resulting therefrom in humans or animals, characterised in that the composition comprises quercetin, or analogues, or derivatives thereof.

2. The composition according to claim 1, wherein said quercetin is in a concentration range of 0.1 to 100% by weight.

3. The composition, according to claim 1, wherein the effective concentration of said composition is in a range of 29 μg/ml (28.9 μg/ml)-35 μg/ml (35.7 μg/ml) of quercetin.

4. The composition, according to claim 1, wherein the composition formulated with a pharmaceutically acceptable carrier.

5. The composition, according to claim 1, wherein the effective concentration of said composition having antiviral activity is 35.7 μg/ml of quercetin.

6. The composition, according to claim 1, wherein flavivirus comprises of dengue virus.

7. The composition, according to claim 1, wherein said composition is for the prophylaxis or treatment of dengue infection.

8. The benefit of the America Invents Act and the first-inventor-to-file provisions.
